(19) United States
(12) Patent Application Publication
(10) Pub. No.: US 2023/0089697 A1
(43) Pub. Date: Mar. 23, 2023

(54) METHOD FOR GENERATING A COMPOSITE NUTRITIONAL INDEX, AND ASSOCIATED SYSTEM

(71) Applicant: FABLIFE, RUEIL MALMAISON (FR)

(72) Inventor: Eric LAMEIGNÈRE, PARIS (FR)

(21) Appl. No.: 17/798,768

(22) PCT Filed: Feb. 11, 2021
(86) PCT No.: PCT/EP2021/053289

§ 371 Date: Aug. 10, 2022

(30) Foreign Application Priority Data
Feb. 11, 2020 (FR) FR2001364

(51) Int. Cl. G16H 20/60; G16B 20/40; G16B 50/30

(52) U.S. Cl. CPC G16H 20/60; G16B 20/40; G16B 50/30

(57) ABSTRACT

A method for generating a composite nutritional index includes selecting an individual; acquiring a first set of phenotypical data for the individual characterizing phenotypic descriptors; acquiring a second set of data for a genotype characterizing genotypical descriptors for the individual; applying a set of predefined rules; generating a set of personalized phenotypical and genotypical indices for an individual; calculating a target value of a daily intake of the at least one nutrient from the application of an inference engine and determining a composite nutritional index including an operation to associate a plurality of target values of a daily intake of the at least one nutrient with at least one metabolic function.

The field of the invention relates to the field of methods and systems intended to generate quantifications of daily nutrient intakes based on an individual's personalized parameters.

Recommendations currently exist to develop reference nutrient intake values for a population of individuals. Current methods generally define values that take account of a few parameters of an individual such as gender, age or condition, for example, a pregnant woman.

However, current methods do not allow classes of individuals with physiological or genetic specificities to be taken into account in order to generate personalized/individualized nutritional recommendations.

A first obstacle is the multitude of factors that can possibly be taken into account. It is difficult to set priorities between factors and to establish causal relationships based on a person's physiological or medical history and the genetics of a subject. Methods aimed at integrating a wider range of parameters even encounter incompatibilities between recommendations that may sometimes be contradictory or non-standard depending on the physiological signs considered and in particular pathological situations.

There is a need to automatically and quickly generate quantifications of nutrient intakes for an individual defining personalized or individualized recommendations. This need should preferably take account of not only a wide range of parameters, but also the specificities of the individual to determine appropriate daily intake values compatible with each other.

There is therefore a need to define a solution that can respond to this problem. The present invention aims to resolve the aforementioned disadvantages.

According to one aspect, the invention relates to a method for generating a composite nutritional index comprising:
Selection of an individual;
Acquisition of a first set of phenotypical data of said individual characterizing phenotypical descriptors, said data comprising at least one age data, one gender data and at least one set of data characterizing physiological signs of said individual;
Acquisition of a second set of data of a genotype characterizing genotypical descriptors of said individual, said data comprising information characterizing mutations and/or variations of at least one gene;
Application of a set of predefined rules comprising:
   at least one first subset of rules aimed at generating at least one phenotypical index starting from a calculation of a score of a quantification of a phenotypical descriptor, said index being normalized;
   at least one second subset of rules aimed at generating at least one genotypical index starting from a calculation of a score of a quantification of a genotypical descriptor, said index being normalized;
Generation of a set of personalized phenotypical and genotypical indices for an individual;
Calculation of target values for daily intakes of a plurality of nutrients from the application of an inference engine configured from:
   a knowledge base comprising a repository of predefined values of phenotypical and/or genotypical indices and at least one set of conditional rules applied to said predefined values of phenotypical indices and genotypical indices; and
   a facts base comprising all phenotypical and genotypical indices of said individual calculated from the data acquired,
Determination of a composite nutritional index comprising an operation of associating a selection of target values of daily intakes of the set of nutrients with at least one metabolic function.

The method is preferably carried out by computer.

One advantage is that daily intakes of nutrients are calculated automatically in a suitable manner and are individualized for a given individual. Another advantage is that these intakes are grouped according to predefined metabolic functions. This grouping makes it easier for the individual to read. Grouping target values for daily intakes together also improves the individual response to be provided for each metabolic function. Another advantage is the synergy of metabolic effects obtained when the daily intakes are monitored by an individual.

Finally, the use of an inference engine in this case allows a convergence of multi-criteria values, each criterion having dependencies as a function of other criteria. Advantageously, the invention makes it possible to take account of criteria corresponding to genotypical and phenotypical data by eliminating the need for a data model linking the different phenotypical and genotypical factors with each other.

According to one embodiment, the method comprises the following steps:
reception of a plurality of individual reference values of a daily intake of a plurality of nutrients;
generation of at least one differential indicator representing an individual reference value for each nutrient and a target daily intake value for said nutrient.

One advantage of the invention is that individualized reference values are taken into account. The individualized reference values take account of a phenotypical and genotypical context of the individual. One advantage over existing solutions is that adaptation of target values is optimized for a given individual.

According to one embodiment, the individual reference values for daily intakes of said nutrients are:
directly extracted from a knowledge base referencing predefined daily intakes of nutrients and/or;
calculated from reference rules automatically calculating daily intakes of nutrients from phenotypical data for said individual and predefined values referenced in a knowledge base.

According to one embodiment, the step to determine a composite nutritional index includes a plurality of groupings of target values of daily nutrient intakes, each grouping contributing to improving a given metabolic function of said individual.

One advantage is that a recommendation for intakes for a given metabolic function can be provided. This grouping makes it possible to pool an action by a user aimed at responding to a metabolic function. Finally, this solution allows optimization of a nutritional action to achieve a metabolic objective.

According to one embodiment, nutrients are macronutrients or micronutrients, said macronutrients being associated with a metabolic function quantifying an energy intake of said individual.

According to one embodiment, at least one reference value of a daily intake of a global quantity of energy of at least one macronutrient is calculated for an individual from a first set of phenotypical descriptors including age, a gender. In this case, at least one target value of a daily intake of a global quantity of energy of said macronutrient is calculated for said individual from a first set of phenotypical descriptors comprising an age, a gender and a second set of phenotypical and/or genotypical descriptors.

According to one embodiment, at least one reference rule includes at least one operation manipulating a first set of quantifications of phenotypical descriptors, for the calculation of a reference value of a daily intake of a given nutrient. In this case, at least one target rule includes at least one operation manipulating a second set of quantifications of phenotypical and/or genotypical descriptors for the calculation of a target value of a daily intake of the nutrient, in addition to the first set of quantifications of phenotypical descriptors.

One advantage of this modeling is to compare a target value calculated by a target rule taking account of different criteria with a reference value obtained by a reference rule. As a result, reference values can also be customized to optimize individualization of the calculation of target values.

According to one embodiment, at least one reference rule includes at least one operation considering a first set of quantifications of phenotypical descriptors, for the calculation of a reference value of a daily intake of a nutrient. In this case, at least one target rule includes at least one operation aimed at defining a fixed value of a daily intake of said nutrient based on at least one threshold value reached by at least one quantification of a phenotypical and/or genotypical descriptor, for the calculation of a target value of a daily intake of the nutrient.

One advantage is that a threshold value is taken into account in order to generate a target value that is still acceptable from the point of view of a nutritional recommendation. This solution can also be used to generate target values of nutritional supplements when a threshold is reached.

According to one embodiment, at least one phenotypical descriptor is calculated from a sum of scores, each score quantifying a physiological condition of the individual.

One advantage is the indices generated can be quantified and standardized to carry out transactions, particularly on a wide range of users.

According to one embodiment, at least one step to generate a list of recipes is carried out, said list of recipes being extracted from a database of recipes comprising a set of recipes each containing a list of ingredients, each ingredient being associated with a list of macronutrients and micronutrients, each of said nutrients being quantified for a recipe according to a value and at least one time data quantifying a time period during which the nutrients are present in the body, said extraction operation correlating target values of daily intakes of nutrients with the recipe base in order to produce a list of recipes for a plurality of days.

One advantage is that a diet program is automatically determined that responds to target daily intakes that can be smoothed over a predefined period of time.

According to one embodiment, the recipe base is filtered from a selection of predetermined ingredients, said recipes generated in the list not comprising the filtered ingredients.

According to another aspect, the invention relates to a computer program product comprising a calculator and a memory, said program comprising program code instructions executed on a computer for implementation of the steps of the method according to the invention.

According to another aspect, the invention relates to a system comprising at least one calculator, a memory and an interface to implement the method according to the invention. The system according to the invention advantageously includes at least one terminal or computer of a user to record the data acquired by means of an interface. In addition, the system according to the invention advantageously includes at least one communication interface to transmit the acquired data to at least one remote server. The system includes at least one remote database and a remote calculator, such as a remote server, enabling operations to generate customized phenotypical and genotypical indices, target values for daily intakes of a plurality of nutrients, and a composite nutritional index.

According to one embodiment, the system according to the invention comprises a memory to store a repository comprising at least predefined data for thresholds, ranges of values, scale of values and predefined calculation rules, the system also comprising a data acquisition interface for a first and a second data set for at least one individual and a memory for storing said data, the system comprising a calculator to execute a set of rules and an inference engine to produce a composite nutritional index making use of the method according to the invention, the system also comprising a display for displaying said composite nutritional index.

Other features and benefits of the invention will be given in the following detailed description, with reference to the attached figures; that illustrate:

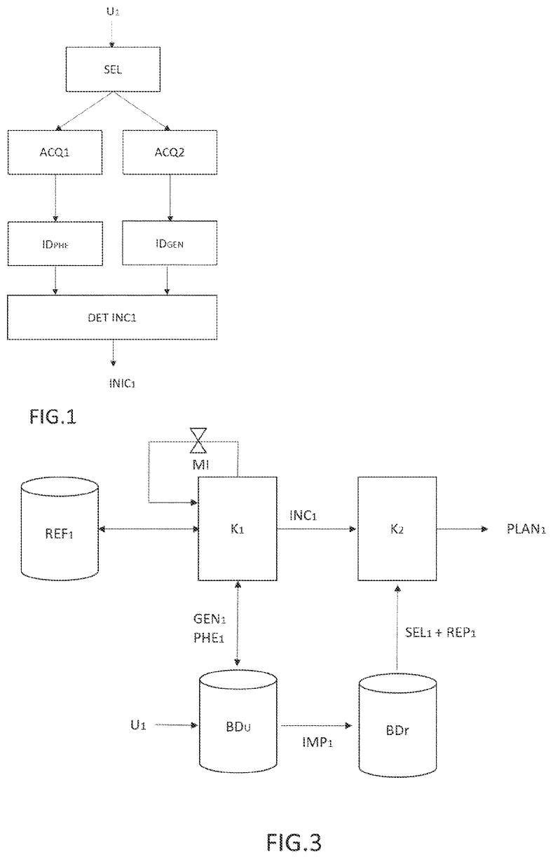

FIG. 1: the main steps of a method of carrying out the method according to the invention:

FIG. 3: an example of processing the data acquired from the method according to the invention in order to generate normalized input data to the inputs of the inference engine,

FIG. 3: an example of a system architecture according to the invention.

### DEFINITIONS

In this description, a phenotype includes all objective and quantifiable data for an individual, such as his or her age, weight, height, etc., and condition data that can be collected and processed by the method and system according to the invention. Condition data comprise particularly data describing physiological signs such as symptoms and data relating to a physiological condition of an individual or a physiological activity. Physiological condition data or physiological activity data may result from the acquisition of information to describe an individual's habits, health practices and/or lifestyle. Phenotypical data are subject to change over time and can be updated during the method in order to generate an updated composite nutritional index. Therefore, for a given subject, the nutritional index is an index dependent on the “time” variable.

A phenotypical descriptor is objective data or condition data for a phenotype that can be quantified. The quantification operation can be simple when the descriptor is already processing quantified data such as anthropometric data for an individual. For example, this may include age, weight, height. The quantification operation can be more complex and be the result of a mathematical operation, such as the calculation of the Body Mass Index (BMI). This operation usually manipulates basic phenotypical data that have already been quantified, such as the height and weight for the BMI. Finally, the quantification operation may correspond to an operation aimed at qualifying and measuring the manifestation of a physiological sign relating to a physiological activity, or to a biological sign of an individual, on a predefined scale. Phenotypical descriptors may also include one or more levels of biomarkers taken from different biological fluids of an individual or more broadly a measured or acquired biological constant for an individual. The quantification of descriptors may be modified over time by various successive measurements. Thus, a variation in the quantification of a descriptor can also constitute a new quantification of a descriptor. For example, a weight reduction can be quantified. The decrease in blood pressure over time is also quantified. Other characteristics corresponding to variations in a quantification may constitute quantified magnitudes of a descriptor. Variations are preferably quantified over predefined time periods.

When the quantification of a value is normalized on a predefined scale of values, this is also referred to as a “qualified value”.

Data characterizing phenotypical descriptors are specially formatted for storage in a physical memory. For example, the memory may be a database with an architecture that allows the use of data characterizing these descriptors. Thus, data such as weight, BMI, age, height, gender, a biological constant, biomarker values, etc., may preferably reflect the architecture of the database used such that these data can be extracted and used by one of the steps in the method according to the invention during calculations.

A phenotypical index, denoted IP_{N}, is a normalized value of a quantification of one or more phenotypical descriptors, that may or may not be combined. Phenotypical indices are calculated from a scale of predefined values. The scale of values is defined in a given knowledge base repository. In the context of the invention, said scale of values is generally associated with conditions aimed at discriminating the result of at least one rule applied to a phenotypical index, by means of an inference engine. When different phenotypic descriptors are grouped within the same phenotypical index, one or more rules can be applied to generate a phenotypical index score based on a plurality of quantifications of phenotypical descriptors. In special cases, the phenotypical descriptor and the phenotypical index may be identical.

In this description, a genotype includes all data describing all or part of an individual's genetic capital. In particular, the sequence of a selection of an individual's genes is exploited when implementing the invention. Therefore, within the scope of the invention, the genotype may designate the sequence of a selection of genes as well as a description of said genes, referring in particular to the related genetic variations or mutations.

A genotypical descriptor is genotype data that can be quantified or qualified. The quantification operation may or may not include the identification of genetic mutations, for example genetic variations, and their enumeration.

Genetic mutation may be one-off or relative to a variation in the sequence of a gene involving several phenomas. A variation in the sequence of a gene can be of different types, and particularly:
mutation by substitution, corresponding to replacement of one (or several) nucleotides with another (or several other) nucleotides;
mutation by insertion, corresponding to the addition of one or several nucleotides;
mutation by deletion, corresponding to a loss of one or several nucleotides
mutation by inversion, corresponding to a permutation of two neighboring deoxyribonucleotides.

A genotypical descriptor may also qualify and quantify a polymorphism in which variability is observed in the number of copies of the same gene or a chromosomal segment in the genome, also known as “copy number variation”, for which the acronym is CNV.

The data characterizing genotypical descriptors are specially formatted for storage in a physical memory. For example, the memory can be a database for which the architecture allows the use of data characterizing the descriptors. Thus, mutations, their number, their type, etc., may preferably reflect the architecture of the database used so that this data can be extracted and used in the calculations in one of the steps of the method according to the invention.

A genotypical index; denoted IG_{N}, is a normalized value of a quantification of a genotypical descriptor, therefore it can be a qualified value. For each copy of the genome present in each individual, genotypical indices are calculated from a scale of predefined values referring to predefined states such as a “mutated” state or a “wild” state of a gene. According to one embodiment, in addition to the determination of a “mutated” or “wild” state, the genotypical index may incorporate the more or less deleterious consequence on the physiology of the individual. The scale of values is defined in a given knowledge base repository. Within the framework of the invention, said scale of values is generally associated with conditions aimed at discriminating the result of at least one rule applied to a genotypical index, by means of the inference engine.

In some special cases, the genotypical descriptor and the genotypical index are identical.

An inference engine is a computer program, software or application that uses a deductive reasoning simulation algorithm. In particular, deductive reasoning can generate converging conclusions of results at the output from the inference engine, firstly from a facts database comprising data for individuals, and secondly a knowledge base comprising a set of rules, conditions and a repository of predefined values.

Known inference engines include engines such as CLIPS, GEOMETRIX, PROLOG, KADVISER, etc. The invention is not limited to a given implementation of an inference engine. Any type of compatible inference engine for operations implemented by the application of rules applied to N inputs with P outputs may also be implemented in the method according to the invention.

In the remainder of the description, a repository is a subset of data in the knowledge base containing all reference data as well as predefined rules.

For example, this data may include
predefined thresholds, so as to produce one or more comparable values when a rule is intended to compare an input variable with a threshold,
limiting values delimiting the framework for the definition of a variable,
ranges of values, for example defining a scale of values to determine a normalized value,
reference daily intake values when used directly,
absolute values for comparing phenotypical or genotypical index values.

The predefined rules of the knowledge base comprise rules that can be played to:
quantify phenotypical descriptors starting from responses to questionnaires, or;
consolidate physiological or biometric values starting from predefined calculation rules, or,
normalize values of phenotypical or genotypical descriptors, that for example compile different condition values.

Finally, other rules of the repository are used when running the inference engine and are applied to data in the database relating to a new subject.

Certain rules, known as reference rules, can be used to calculate reference values for each new individual starting from the repository. Other rules, called target rules, can be used to calculate target values for each new individual staring from the repository and the inference engine.

A composite index representing a target value associated with a reference value can then be generated.

Profile, Phenotype

The method according to the invention makes it possible to receive a first data set ENS₁ and a second data set ENS₂ from a user. The first data set ENS₁ characterizes data specific to the user's phenotype PHE₁. The second data set ENS₂ characterizes data specific to the user's genotype GEN₁. These data sets ENS₁ and ENS₂ can be acquired in a single step. The sets ENS₁ and ENS₂ are functionally differentiated, for example for the processing of acquired data, but may be inseparable for the user who will deliver these data from a system interface such as that generated by a computer.

The first data set ENS₁ comprises data that can be entered via a human-machine interface, known as the HMI, that can be used to configure data specific to a user. According to one example, some data from set ENS₁ are automatically acquired from a communication interface through which data can be received and decoded. According to one example, data are automatically acquired from a smart object. Examples include a smart watch, a connected blood pressure monitor, a connected weigh scale or a connected blood glucose monitor.

Different acquisition methods can be used in the invention. For example, data can be received from a wired or wireless interface. The data may be stored on a remote server. Alternatively, the data may be stored in a memory of the same equipment that performs the calculations, so that the steps in the method according to the invention can be performed.

Data in set ENS₁ describing the phenotypical data of PHE₁ for a user includes, for example, profile data for an individual such as age or gender, i.e. gender/sex {WOMAN, MAN}, height, or possibly cultural or residential characteristics. Data in set ENS₁ also includes anthropometric and physiological data for an individual such as weight, or one or more concentrations of biomarkers taken from different biological fluids or more broadly any measured or acquired biological constant for an individual. Data in set ENS₁ may also include data characterizing muscle mass, heart rate, data output from an electroencephalogram, body fat, water content, bone mass, blood sugar, cholesterol, triglycerides, etc.

According to one example, the method according to the invention can be used to categorize phenotypical data into subsets so as to facilitate the acquisition and exploitation of data. For example, the set ENS₁ may include behavioral phenotypic data, emotional or stress phenotypical data, biological phenotypical data, physiological phenotypical data.

Genotype

The second data set ENS₂ comprises data collected after an analysis of the genome of an individual's gene selection, the genotype.

When these data are digitally accessible, a system communication interface is configured to receive the data in digital format. According to one alternative, the data for the second set are recorded via a system interface.

According to one example embodiment a selection of 20 to 30 genes is processed to extract information characterizing the individual state of each gene. In one preferred mode, a data set is extracted, said data characterizing each gene of the selection. A first data G₁ is used that counts genes that have at least one mutation or variation. A second data G₂ may also be exploited when it characterizes a number of modifications of a gene, in other words. a number of mutations or variations of a gene. In this case, it is considered that a modification of a gene corresponds to a modification of generic information, in other words a variation of the sequence. Finally, a third data G₃ describing the type of mutations or variations can be used depending on the embodiment of the invention.

According to one embodiment, rules comprising conditions on a set of mutations or variations of a set of genes can be used to produce an additional fourth data G₄.

Knowledge base rules are configured so as to produce scores depending on the first, second, third and fourth data values G₁, G₂, G₃, G₄ of a gene or a group of genes in the selection.

The method according to the invention can be used to verify the type of genetic mutations such as mutations by substitution, by insertion or by deletion or the variability of the number of copies of the same gene or chromosomal segment. According to one embodiment, the invention also includes an analysis of types of mutations such as duplication, translocation or inversion. The method according to the invention can also take account of the presence of genes of an individual in the homozygous or heterozygous state and the number of associated mutations.

According to one example embodiment, genotypical indices include a normalized quantification for each individual mutation considered among the following three cases:
0 mutations, also denoted wild-type/wild-type homozygous,
1 mutation, also denoted as wild-type/mutant heterozygous,
2 mutations, also denoted as double mutant homozygous).

For example, this quantified value can be associated with a qualitative of the mutation such as a polymorphism type variation of a single nucleotide, known as PSN, or a deletion, insertion or variability of the number of copies.

The method according to the invention comprises a step to describe these data characterizing a genotype of a predefined selection of genes of an individual. To this end, genotypical descriptors can be used to quantify the various data G₁, G₂, G₃, G₄, etc., or even to generate a score characterizing this genotype information depending on the knowledge base.

The method then comprises a step to generate a genotypical index IGᵢ. In particular, the genotypical index IGi is used as input data to the inference engine in the same way as the phenotypical indices IPᵢ. The genotypical index IGi is a normalized value that can be exploited by inference engine rules.

The transition from genotypical descriptors to phenotypical indices can be made by the application of predefined rules, said rules being applied before execution of the inference engine. For example, the presence of one or more mutations in a particular person can be used to modulate the person's phenotypical profile through the application of one or more rules.

Rules

The method according to the invention includes a database in the knowledge base, in which rules are recorded and possibly updated. The rules define operations to check conditions on the data of sets of the first set ENS₁ and the second set ENS₂.

Certain rules R_{REF} are applied to calculate daily nutrient intakes NUTᵢ and individual reference values V_{IR}, other rules R_{CIB} are applied to calculate target values V_{CIB} of daily nutrient intakes NUT_{I} that correspond to a personalized intake for an individual according to the data in the first and second sets ENS₁, ENS₂.

According to a first case, certain rules R_{CIB} that can be used to calculate target values V_{CIB} are defined based on rules R_{REF} and enrichment of said rules R_{REF}. For example, enrichment includes taking account of an additional factor or a weighting coefficient calculated by taking account of a phenotypical descriptor and/or a genotypical descriptor.

In order to illustrate this example, the method according to the invention can be configured to define a rule R_{CIB} that consists of applying an incremental or subtractive value to a reference score of either a phenotypical descriptor, i.e. a genotypical descriptor, or a phenotypical index, i.e. a genotypical index. For example, the incremental or subtractive value is considered when at least one condition is satisfied.

According to one example, this incremental value may be an additional percentage of a reference daily nutritional intake. V_{CIB}=1.2·V_{IR} is obtained when an additional daily intake of a nutrient must be 20% higher than a reference value V_{REF} of this intake. In this example, the personalized daily intake called the target intake, is calculated independently of other values of phenotypical and genotypical indices at the input of the inference engine.

In particular, according to another variant, the method according to the invention makes it possible to consider a modification of the phenotypical or genotypical index depending on a reference value of the index considered, for example by incrementing this value. Said new value of the phenotypical index is then used by the inference engine, that can converge towards a new daily intake V_{CIB} for a class of nutrients. In the latter case, there is not always an independent causal relationship between the value of the phenotypical index and the value of a daily intake of a nutrient.

Furthermore, according to certain embodiments, the method according to the invention includes scenarios in which values of reference phenotypical and genotypical indices are calculated from a subset of phenotypical or genotypical descriptors. These reference indices can be used to calculate values of reference daily nutrient intakes. One contribution of the invention is the use of an inference engine to calculate target values of daily nutrient intakes that are different from reference values of daily nutrient intakes.

According to one embodiment, descriptors are used to quantify other phenotypical or genotypical descriptors. The term used is consolidation rules. In the latter case, the quantified values of the descriptors are derived from a calculation and are therefore consolidated in that the value of the phenotypical descriptor or the phenotypical index used is calculated from an operation manipulating data acquired for a subject.

According to one example, for a man over the age of 18, an individual reference value V_{IR} (GR, AGE) of a given nutrient NUTi, such as the daily intake of chromium, is calculated as a function of these two parameters: Age: AGE and gender: GR. Numeric charts, ranges of values or thresholds prerecorded in a memory can be used to apply predefined rules.

The calculation of a target value V_{CIB}, corresponding to a daily intake of a nutrient and therefore to the individual reference value V_{IR} of the same nutrient, may take account of a wider range of phenotypical descriptors and genotypical descriptors. According to the example for which a reference value has been previously calculated, the method according to the invention can be used to calculate a personalized target value V_{CIB} V_{CIB} (GR, AGE, BMI, OB, SPO, CHLO, ANX)=V_{IR}+/−10.

In this example, GR denotes gender, AGE denotes age, BMI denotes body mass index, OB denotes an obesity category, SPO denotes a quantification of daily exercise, CHLO denotes a quantification of a cholesterol content, and ANX denotes a quantification of an individual's level of anxiety. These variables are phenotypical descriptors or indices depending on the case.

In this example, the inference engine allows all parameters to be taken into account and to be played to determine a value V_{CIB} that is the result of a combination of causes quantified by the aforementioned descriptors.

In a second case, the rules R_{CIB} are defined by an algorithm that can be used to calculate a target value V_{CIB} based on phenotypical and genotypical indices. This algorithm can be established so that the target value does not result directly or indirectly from an rule R_{REF}, but from a new rule used to calculate individual reference values V_{IR}. For example, a rule R_{CIB} relates to the generation of a fixed value of a daily intake of a nutrient when one or more conditions are satisfied. For example, the condition may be the consideration of a parameter of the individual's phenotype or genotype.

To illustrate this example, one rule R_{CIB} consists of applying a fixed value of a daily quantity of a given nutrient when an individual's phenotype indicates that the individual is a pregnant woman. In this case, the “pregnant woman” parameter corresponds to a phenotypical descriptor or a phenotypical index such that a condition can be generated on the result of a calculation rule and can be used to generate a value of a daily intake of a nutrient.

The method according to the invention is based in particular on a prior configuration step aimed at determining quantifications of ranges of values of phenotypical and/or genotypical descriptors. These quantifications can be used to define conditions for associating results obtained by the application of calculation rules R_{REF} or R_{CIB} to calculate individual reference values V_{IR} or target values V_{CIB}.

These quantifications are preferably normalized by transposing the quantification of ranges of values of descriptors to indices in order to homogenize processing using different rules, making use of an inference engine.

Quantification of Phenotypical Parameters

According to one example embodiment, a first family of quantifications of phenotypical descriptors is calculated from values acquired or received and used directly for an individual. An individual's height and/or weight and/or waist circumference may be examples of descriptors in this quantification family. These values are directly used by rules as a function of other predefined values in the repository. For example, the individual is a man with a BMI higher than a first threshold and with a cholesterol level higher than a second threshold, and in this case a rule can be used to define a value with reference to a list of daily intakes of a subset of nutrients.

According to another example, a second family of descriptor quantifications results from the acquisition of a set of acquired physiological conditions of an individual that are processed so as to generate a physiological quantification. This physiological quantification can be used to generate a quantification of the descriptor or a quantification of a phenotypical index that can be used by other calculation rules of the inference engine. For example, a quantification of a physiological state of fatigue can be generated from a list of answers to predefined questions, for example including a quantification of a volume of sleep, an assessment of daily stress, an assessment of daily physical exertion, a quantification of a level of drowsiness, etc.

According to one example embodiment, values quantifying certain physiological conditions such as stress are normalized on a scale of values comprising a number of predefined values to generate a phenotypical index that can be used by the inference engine.

### Example of Quantification of a Sporting Activity

According to one example, a list of questions can be used to assess a composite score of a physiological condition, on a scale. Each answer to a question increments the composite score. For example, a first question generates an evaluation from 0 to 10, a second question generates an evaluation from 11 to 20 and so on, up to the tenth question, that generates an incremental evaluation from 91 to 100. For example, the value of the incremented composite score is 56/100. Such a composite score can then be normalized on a scale of 0 to 3 or a scale of 0 to 5, depending on the range of values defining the scale of the phenotypical index. One advantage is that physiological conditions are normalized and can then be treated by homogeneous processing in the inference engine. Another advantage is that the variation of the calculated score can be compared dynamically.

According to one example, the assessment of a physiological condition related to a sports activity may include a list of questions to assess the type of sport, its frequency, intensity, etc. The process includes a step to transpose descriptor scores into a predefined scale, so as to determine an individual's sporting activity factor, and to quantify and normalize it. For example, the level of sporting activity can be quantified among 3 values on a normalized scale.

According to another example, a quantification of an individual's psychological stress, a quantification of an individual's health, a quantification of an individual's sense of well-being and morale, a quantification of fatigue, a quantification of the quality of memory and the concentration and quantification of oxidative stress can be assessed. The evaluations include a method for calculating a score, for example an incremental method. This method makes it possible to take account of various factors for which the quantified effects are cumulated. A normalized value can then be calculated to determine an input to the inference engine.

Inference Engine

According to one embodiment, the calculation of a given target value V_{CIB} of a daily intake of a nutrient is obtained from the application of a set of rules applied to a set of values quantifying an individual's phenotypical and/or genotypical indices. In such a case, a plurality of rules aims to perform operations that validate or invalidate conditions, such as exceeded thresholds, lowered thresholds, values within predefined ranges, or monitoring of condition values, etc.

Conversely, the result of an applied rule can influence a plurality of daily intakes of a given set of nutrients.

The model according to the invention results in a model comprising a number N of inputs comprising processing of a plurality of values quantifying phenotypical and genotypical indices for an individual and generating a number P of outputs of this model corresponding to a plurality of daily intakes of individualized nutrients.

According to one embodiment, the invention includes the application of an optimization function aimed at determining one or more application sequences of the inference engine in order to obtain target values V_{CIB} of the different converging personalized daily nutritional intakes with a limited calculation time and cost.

According to one example, the rules can be applied in a given order and the values obtained from the daily intake of a given nutrient are derived from the application of said set of rules according to the first rule application schedule. According to one example embodiment, all the rules are applied according to a second schedule so as to infer a target value of a plurality of daily intakes. The set of rules used to calculate target values V_{CIB} of daily nutrient intakes can then be reapplied a certain number of times until a set of target values V_{CIB} is inferred, optimized with regard to each other and with regard to all rules used. In this case, the set of target values V_{CIB} generated is the result of a convergence of a set of applied rules such that the effects of dependencies of rules on each other and with regard to calculated target values are minimized.

Different optimization functions can be configured to define an inference engine. In particular, the use of an inference engine is based on a facts base and a knowledge base. The knowledge base is the database containing rules and conditions for determining criteria for affiliation of the calculated values. The facts base contains entries corresponding to quantified values derived from an individual's phenotypical and/or genotypical descriptors.

For example, the inference engine can take account of different quantifications of indices derived from phenotypical and/or genotypical descriptors derived from different physiological and genetic parameters to determine daily intakes of macronutrients, such as lipids, proteins and carbohydrates. The different physiological parameters influencing the distribution value of daily intakes of macronutrients are taken into account in the inference engine in order to converge towards optimized values of daily intakes.

A list of daily intakes of nutrients, macro- and micronutrients, is generated at the output from the inference engine. The invention can be used to group these different intakes by major metabolic function. Thus, a first metabolic function includes in particular energy metabolism, a second function relates to lipid metabolism, a third function relates to metabolism of amino acids, a fourth function includes oxidative equilibrium functions, and finally a fifth function relates to quality of life functions.

This categorization is detailed below through example embodiments, however other categorizations can be implemented using the method according to the invention. In particular, the categories of major metabolic functions can be organized and configured according to a given indication such as fertility, sleep, physical recovery and/or physical activity, etc.

For example, the first metabolic function includes the metabolism of macronutrients such as carbohydrates, lipids, proteins, vitamins B1 and B3, target daily intake values V_{CIB} and individual reference values V_{IR} for each of these nutrients.

For example, the second metabolic function includes the metabolism of saturated fatty acids, oleic acid, amino acids, linolenic acid, omega-3s such as EPA or DHA, target values V_{CIB} of daily intakes and individual reference values V_{IR} of each of these nutrients.

For example, the third metabolic function includes the metabolism of vitamins B2, vitamins B6, vitamins B9, vitamins B12, target daily intake values V_{CIB} and individual reference values VIE for each of these nutrients.

For example, the fourth metabolic function includes the metabolism of vitamins A, vitamins C, vitamins E, selenium and zinc, target daily intake values V_{CIB} and individual reference values VIE for each of these nutrients.

For example, the fourth metabolic function includes magnesium and vitamins D, target values V_{CIB} for daily intakes and individual reference values V_{IR} for each of these nutrients.

One advantage of this association between each subset of nutrients grouped together with a metabolic function is to create an appropriate recommendation benefiting from synergy of effects and metabolic coherence. These grouped nutrient subsets can be reconfigured according to a given indication, in other words a fertility, sport or another indication. In another configuration, these groupings and associations produce a different adapted synergy that produces recommendations adapted to the indication.

Finally, each subset can be displayed by means of a graphical interface so as to present composite indicators comprising the individual reference value V_{IR} with which the target value V_{CIB} is associated on a common scale of values. One advantage is that a personality recommendation can be represented for a given individual with regard to a non-personalized reference value.

Food Recipes

The invention can be used to proposed a set of recipes to an individual, making use of the composite indicator thus generated, “Recipe” is understood to mean a dish or a set of ingredients making up a dish.

According to one embodiment, the system according to the invention includes a recipe database. For example, each recipe includes an identifier, a name, a list of ingredients, each ingredient is associated with its nutritional value depending on said ingredients, the quantity of ingredients and possibly the preparation such as the cooking method.

Each ingredient for a given quantity can be segmented into a quantified list of macronutrients, micronutrients and calories that it contains or provides.

Conversely, knowing the composition of nutrients of a plurality of ingredients and therefore recipes, the method according to the invention can be used to. generate a set of recipes for an individual, based on a daily nutrient recommendation.

The method according to the invention includes a calculation step to optimize the distribution of recipes over a given period, for example a period corresponding to “the week”. One advantage of this optimization is that the nutrient intake of the food is “smoothed” depending on the recipes, in other words a nutrient intake is spread over time over a given period. For example, if an iron intake of 10 mg/day is recommended for a man as a function of his phenotype and genotype, depending on the period of digestion, absorption, assimilation and persistence of iron in the body, recipes can be distributed to ensure an average weekly intake corresponding to a week-long recommendation rather than a day-long recommendation. This is also the case for certain nutrients/foods with longer or shorter persistence in the body such as a liposoluble vitamin or another water-soluble vitamin.

According to another example, the intake of vitamin C, a water-soluble vitamin, cannot be stored, which means that it persists for a shorter period of time than minerals such as iron or other liposoluble vitamins. The indicated recipes then include ingredients that make it possible to make a suitable recommendation on a day-to-day basis, as close as possible to the required daily nutrient recommendation. On the other hand, with the example of liposoluble vitamins, the intake of recipes for these nutrients is smoothed over periods of up to several days, and the average daily intake over this period remains the same as that calculated in the previous step.

Therefore, the recipe database BDr includes specific data determining for how long the daily nutrient intake for each nutrient can be considered to generate meal recommendations over a given period. On the other hand, the nutrient intake of each recipe can be determined automatically from a reference database. For example, the French reference base is the CIQUAL base updated by the French Agency for Food, Environmental and Occupational Health & Safety (Anses). Several countries are developing and maintaining their own reference bases. One or more reference databases can be used concurrently to automatically calculate the cumulative quantitative intakes of each ingredient contained in each recipe depending on the country or cultural preferences. For example, this calculation can be carried out by taking account of one or more factors that weight quantities of macroelements. Other criteria can be taken into account. According to another embodiment, a country can be taken into account using another technique.

Individualization of Food Constraints

The method according to the invention makes it possible to take account of certain dietary constraints or habits of a subject. One advantage is to filter ingredients and therefore recipes containing them in the generated recommendation. Dietary constraints can be due to an allergy, intolerance, an individual's taste or cultural well-being. Thus, the recommended recipes include not only a personalized daily nutritional intake but also filtered ingredients or groups of filtered ingredients so that all recipes can be consumed by the individual.

Therefore, the method according to the invention makes it possible to use the recipe database with data stored in a memory corresponding to an individual's personalized profile in order to generate proposals for a list of recipes that take account of the constraints and dietary habits of a subject.

Ingredients comprising a given nutritional value that are filtered due to a dietary constraint are automatically replaced by one or more other ingredients in order to propose a new dish that the subject can consume. The method according to the invention may include a step to filter all recipes from the recipe base BDr comprising ingredients that are set aside by a subject. The recommended nutrient intakes are then provided by recipes that have not been filtered.

Coverage Index

According to one embodiment, the selection of proposed recipes includes a recommended daily nutrient coverage indicator to inform an individual if the recipe is consistent with the generated composite index. One advantage is to provide a list of alternative recipes that are less effective than the recommended main recipe(s). This option gives an individual a choice of alternative recipes that are less optimized, but giving a wider choice. According to one example, the user of the interface can use the interface to choose recipes covering 80% of the recommended daily intake or 90% or even 100% for a strict diet. According to one example, a third party such as a doctor or nutritionist accesses the recipes from the interface and their coverage ratio with regard to the recommendation for a given patient.

Dietary Impasse

When a recommended nutrient intake cannot be provided by the ingredients of a recipe in the database or when too few recipes correspond to a given intake of a nutrient, the method according to the invention can be used to calculate additional nutrient supplements for an individual. Supplementation can be calculated quantitatively and qualitatively, nutrient by nutrient. Supplementation is personalized particularly by a selection of supplements for a given individual. According to one example, the recommended nutrient intake can be considered as “not assured” when this intake is not assured by an averaged sum of daily intakes for a given period. This is the case with vitamin D, which can be added as a supplement to make up for a shortfall over a period of time. The individualized composition of supplements is determined based on a nutrient deficit in the recipes or to ensure variability in dishes to supplement the intake of a given nutrient for a given individual at a given time.

### EXAMPLE EMBODIMENTS

FIG. 1 represents the main steps of an embodiment according to the invention. A selection step SEL is performed for an individual. The selection step includes at least one identification of the subject. Identification can be done by means of an interface through which a name, identifier or any other data digitally linked to an individual's identifier, can be selected. For example, the selection of an individual includes the selection of a digital profile describing the digital characteristics of a predefined individual. The method according to the invention is particularly interesting due to the generation of a list of personalized daily nutrient intakes. As a result, the method is preferably carried out for one individual at a time.

A first acquisition step ACQ₁ is performed to collect phenotypical data for the selected individual. A second acquisition step ACQ₂ is performed to collect the genotypical data for an individual. These steps advantageously collect data organized in the form of descriptors to quantify phenotypical and genotypical data. According to another example, the descriptors are a preliminary calculation step for the determination of phenotypical and genotypical indices. Acquisitions can be made at the same time or at different times.

The method includes steps to generate phenotypical and genotypical indices for an individual from the descriptors. Sometimes, the values of the descriptors correspond to the values of the indices. This is the case when the acquired or processed numerical values have already been normalized. Phenotypical and genotypical indices are the input data for an inference engine MI.

The method involves a step to determine a composite nutritional index DET_INC₁ to produce a composite nutritional index INC₁. This index includes a list of daily nutrient intakes including macronutrients and micronutrients.

The step to determine the composite nutrition index INDC₁ is performed using an inference engine.

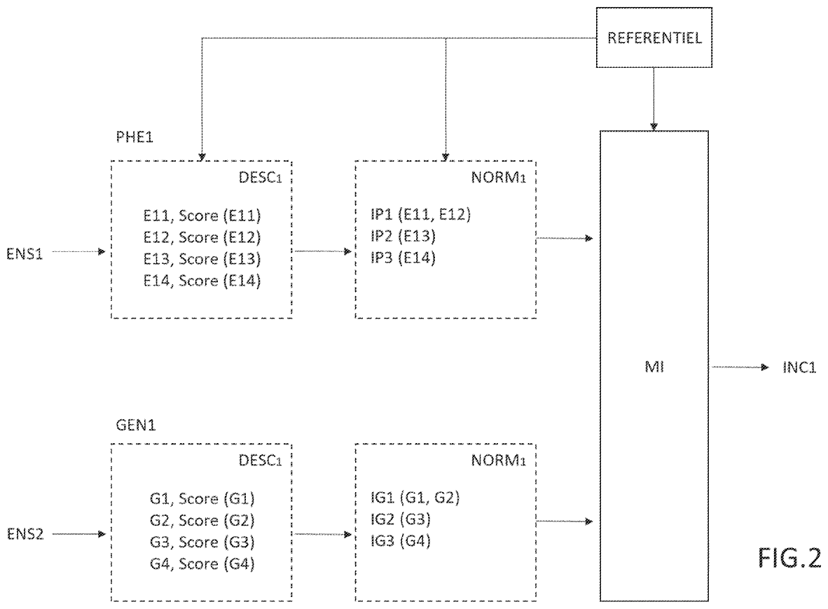

FIG. 2 gives details of an example in which the first set ENS₁ includes four physiological conditions E11, E12, E13, E14. Each of these conditions represents a part of a phenotype denoted PHE₁ of the individual U₁. All four conditions can be processed by rules from a REF₁ repository knowledge base in order to calculate scores associated with the conditions. Scores are denoted Score(E11), Score(E12), Score(E13), Score(E14). In this example, scores are calculated from the conditions in a block denoted DESC₁ in FIG. 1.

In this example, the scores Score(E11) and Score(E12) are used to calculate a phenotypical index IP1(E11, E12), and scores Score(E13) and Score(E14) are used to calculate indices IP2(E13) and IP3(E14) respectively, for example using a calculation rule from the knowledge base. The calculations of phenotypical indices are performed in a block denoted NORM₁ in FIG. 1. It is then understood that descriptor scores can be combined to produce phenotypical indices such as IP1.

Values of phenotypical indices are thus transmitted to a block denoted MI corresponding to the algorithm configured to define the inference engine.

FIG. 1 includes another data processing sequence to receive and process data in set ENS₂ corresponding to genotype data GEN₁ for the same individual.

FIG. 1 includes a first block noted DESC₁ to acquire genotype data G1, G2, G3 and G4. The genotype data are then processed to calculate scores Score(G1), Score(G2), Score(G3) and Score(G4). For example, this could be the number of mutations of a gene, the type of mutation of the same gene, etc.

A second block denoted NORM1 is used to calculate genotypical indices IGᵢ to define inputs to the inference engine M. When Scores(Gi) already correspond to a normalized score, the genotypical index IGᵢ may be identical to the previously calculated score.

The inference engine MI is then applied to all input data, i.e. phenotypical and genotypical indices using rules defined in the repository REF₁. The inference engine MI is used to determine convergent target values V_{CIB} of an individual's target daily nutritional intake.

FIG. 3 shows an example of the architecture of the system according to the invention.

The repository is denoted REF₁ in FIG. 3. It includes at least one database or a file comprising predefined thresholds, predefined value ranges, predefined rules and value scales used for normalizing scores. It may also include reference values such as reference values of descriptors or indices or even individual reference values V_{IR} of daily intakes for typical profiles. The repository REF₁ is used by a calculation block denoted K₁. The calculation block K₁ may consist of one or more calculators, such as microcontrollers, microprocessors or any other means of making digital calculations.

The calculation block K₁ can quantify descriptors using a first level of rules, normalize these values to generate indices using a second level of rules and play the inference engine using a third level of rules. The inference engine is denoted MI herein.

According to one embodiment, a user database denoted DBu, can be used to select a user U₁ stored in said base. Each user profile U₁ may be associated with the phenotype PHE₁ and the genotype GEN₁ of said user U₁, after acquisition of these data.

One of the benefits of capitalizing on a database of BDu users is the generation of a statistical engine, for example, based on artificial intelligence, Such a configuration makes it possible to establish metrics over time specific to successes or failures of recommendations based on the calculation of a composite nutritional index of a set of individuals. Recommendations for a new individual can thus be modulated or adapted depending on the (effectiveness), the success or failure rates of a set of profiles similar to said new profile.

According to one embodiment, the composite nutrition index INDC₁ is calculated at different periods, that may or may not be regular. The advantage of calculating the composite nutrition index INDC₁ at different times is that the individualized recommendations can be adapted by taking account of changes in conditions that may occur after the individual U₁ has followed the recommendation.

A second calculator K₂ is shown in FIG. 3. It can use a recipes base BDr to produce a meals planning PLAN₁ over a defined period of time for a given user U₁. According to one embodiment, the calculator K₂ can be the same as the calculator K₁, depending on the selected system architecture.

The calculator K₂ uses a recipes base BDr, a list of constraints and dietary habits IMP₁ predefined by the user U₁, a database of food supplements (not shown) and rules for diversifying the diet over time, in other words the distribution of nutritional intakes over time scales dependent on data specific to absorption, assimilation and persistence of food in the body.

According to one example embodiment, these rules for diversification of food over time can be integrated into the recipe base BDr in order to enrich recipe data. These rules are noted REP₁ in FIG. 3.

Dietary constraints herein are extracted from the user base DBu that stores user profile and preferences data. In this description, the constraints are denoted IMP₁.

1. A method for generating a composite nutritional index comprising:
selecting an individual;
acquiring a first set of phenotypical data of said individual characterizing phenotypical descriptors, said data comprising at least one age data, one gender data and at least one set of data characterizing physiological signs of said individual;
acquiring a second set of data of a genotype characterizing genotypical descriptors of said individual, said data comprising information characterizing mutations and/or variations of at least one gene;
applying a set of predefined rules by a calculator, comprising:
at least one first subset of rules aimed at generating at least one phenotypical index starting from a calculation of a score of a quantification of a phenotypical descriptor, said index being normalized;
at least one second subset of rules aimed at generating at least one genotypical index starting from a calculation of a score of a quantification of a genotypical descriptor, said index being normalized;
generating a set of personalized phenotypical and genotypical indices for an individual;
calculating target values for daily intakes of a plurality of nutrients from the application of an inference engine configured from:
a knowledge base comprising a repository of predefined values of phenotypical and/or genotypical indices and at least one set of conditional rules applied to said predefined values of phenotypical indices and genotypical indices and;
a facts base comprising all phenotypical and genotypical indices of said individual calculated from the data acquired, and
determining a composite nutritional index comprising an operation of associating a selection of target values of daily intakes of the set of nutrients with at least one metabolic function.

2. The method for generating a composite nutritional index according to claim 1, further comprising:
receiving a plurality of individual reference values of a daily intake of a plurality of nutrients, and
generating at least one differential indicator representing an individual reference value and a target value for daily intake of said nutrient, for each nutrient.

3. The method for generating a composite nutritional index according to claim 2, wherein the individual reference values of daily intakes of said nutrients are:
directly extracted from a knowledge base referencing predefined daily intakes of nutrients, and/or
calculated from reference rules automatically calculating daily intakes of nutrients from phenotypical data for said individual and predefined values referenced in a knowledge base.

4. The method for generating a composite nutritional index according to claim 1, wherein determining a composite nutritional index includes a plurality of groupings of target values of daily nutrient intakes, each grouping contributing to improving a given metabolic function of said individual.

5. The method according to claim 1, wherein the nutrients are macronutrients or micronutrients, wherein said macronutrients are associated with a metabolic function quantifying an energy intake of said individual.

6. The method according to claim 5, wherein:
at least one reference value of a daily intake of a global energy quantity of at least one macronutrient is calculated for an individual starting from a first set of phenotypical descriptors comprising an age, a gender, and
at least one target value of a daily intake of a global energy quantity of said macronutrient is calculated for said individual starting from a first set of phenotypical descriptors comprising an age, a gender and a second set of phenotypical and/or genotypical descriptors.

7. The method according to claim 2, wherein:
at least one reference rule includes at least one operation manipulating a first set of quantifications of phenotypical descriptors, for the calculation of a reference value of a daily intake of a given nutrient, and
at least one target rule includes at least one operation manipulating a second set of quantifications of phenotypical and/or genotypical descriptors, for the calculation of a target value of a daily intake of the nutrient, in addition to the first set of quantifications of phenotypical descriptors.

8. The method according to claim 2, wherein:
at least one reference rule includes at least one operation considering a first set of quantifications of phenotypical descriptors, for the calculation of a reference value of a daily intake of a nutrient, and
at least one target rule includes at least one operation aimed at defining a fixed value of a daily intake of said nutrient based on at least one threshold value reached by at least one quantification of a phenotypical and/or genotypical descriptor, for the calculation of a target value of a daily intake of the nutrient.

9. The method according to claim 1, wherein a phenotypical descriptor is calculated from a sum of scores, each score quantifying a physiological condition of the individual.

10. The method according to claim 1, further comprising generating a list of recipes, said list of recipes being extracted from a recipe database comprising a set of recipes each containing a list of ingredients, each ingredient being associated with a list of macronutrients and micronutrients, each of said nutrients being quantified for a recipe according to a value and at least one time data quantifying a time period during which the nutrients are present in the body, said extraction operation correlating target values of daily intakes of nutrients with the recipe base in order to produce a list of recipes for a plurality of days.

11. The method according to claim 10, wherein the recipe base is filtered from a selection of predetermined ingredients, said recipes generated in the list not comprising the filtered ingredients.

12. A system comprising a memory to store a repository comprising at least predefined data for thresholds, ranges of values, scale of values, and predefined calculation rules, the system also comprising a data acquisition interface for a first and a second data set for at least one individual and a memory for storing said data, the system comprising a calculator to execute a set of rules and an inference engine to produce a composite nutritional index using the method according to claim 1, the system also comprising a display for displaying said composite nutritional index.
